SEQUENCE LISTING (3 sequences)

SEQ ID NO: 1 - protein, 547 aa, Oscillatoria nigro-viridis
TRDTSRDGFSNKALAYTLTHFKPIWNLVQSYEPLKRKLNKFFLNSIIYKLPTRPLPYSLMGLDPKIPGTDIPKKTDTYISWDSLTDKTYTGRHLPPDPEFNKEGNLPPLDKVKTLFQKRDGKTIYSEKSTLLFPYWVQWFTDSFLRIDQENRFKNTSNHQIDMCNVYGLTRKQTNMLRAFKDGKFKTQKLKRKDGVEEEYPLFYYADPEQGIIDPQFEGLHAPLNDEKRQPPEKKSKLFAMGVERANVQIGYVMLNTLCIREHNRICDVLSKSYPEWDDERLFQTARNILMVIVLNIIMEEYIFHITPYNFRFFADPEAFTKESWYRENWMAIEFSFVYRWHSAIPETFIYDGKEQSMYDSLWNNQMLIDKGLGALMEETCSQPGTRIGLFNTPDFKIAGTPYTFIDATELASVKLGRQAQLASYNDYREMCGYPRVTDFNQITGDEYAQQKLKELYGHVDKIELFVGLYAEDVRKNSAIPPLVARIIGIDAFSQALTNPLLSPKVFNKETFSEVGWEIIQNTKTVSDLVNRNVPPSDPKYKVSFEL

SEQ ID NO: 2 - protein, 540 aa, Synechococcus PCC7509
RDTSKDGFRNKLETYALTHFKPIWNLIQSNDTLKKKVNKFLVNNAIYKVPTRPYPFSTMSPYTSWDSLSDRTYSGLHLPPLDWQPLTNENHLKLKLADTKDFEKKLPAIEDLRGLYRKSGETKYSPKSTLIFPYFVQWFTDSFLRTDRHNHRKNTSNHQIDLCTVYGLNAKITHLLRSYQGGKLKSQIINGEEYPPFYYDEKGEAKKEFIGLPHQLDNDGNPKADTFPLDKKQKLFAMGVEVERSNVQIGYVMLNVLALREHNRLCELLAKTYPSWDDERLFQTARNILIVEVLRIVVEDYVNHITPYHFQFITDPLTFSNEKWYRQNWMTVEFTLVYRWHSMLPDTLIYNGQKIPTYETQWNNEMIIKQGLGALFEESCSQPCAQLSLFNTPEFLIPVELASVRFGREVKLRSYNDYRQLCKYPRVTDFDQISSDKNIQKELQRLYGHVDNIELYVGIYAEDLRENSALPSLVGRLIGIDAFSQVLTNPLLAESVFHPETFSPVGWEEIQNTKTLSQLLHRNLPPSDKKYRVSFDRAST

SEQ ID NO: 3 - protein, 543 aa, Tolypothrix campylonemoides
AGKRDTSKDGFDNKVQTFLLTNFKGIWEIVQSNEFLKRKVNKTLINSLIYKIPTRPNPYSMMTLDEYIPDTKIPKKTDTYTSWELLNDRTYIGRHLPPDPKFNSEGNLPKVEDLAVLFRKRDGKTIYSPKSTMLFPYWVQWFTDSFLRIDHTKEKKLKNTSNHEIDLCNVYGLNRKRTHLLRTFKGGKFKTQKLKRQDGIEEEYPLFYYADPAQGIVDPQFDGLYEPINDEKRLPADKKQYLFAMGVERANVQIGYVMLNTLCIREHNRLCDELASNYPDWDDERLFQTSRNILMAIILNIIMEEYINHITPYHFKLFADPAAFVKESWYRPNWMTIEFDFVYRWHSAIPETFIYDGQPTDIAASLWNNKMFIDKGLGALMEETCSQPGTRIGLFNTPDILVELTELPSIRLGRQLQLASYNDYREMCGFPRVTKFEQITGDEFAQEKLKELYGHVDNIEFYVGLYAEEVRKNSTIPPLVARLIGIDAFSEALNNPLLSPTIFNKDTFSPVGWEIIQNTKTVSDLINRNVPPSDKKYKVTFDL

(19) United States
(12) Patent Application Publication
(10) Pub. No.: US 2018/0077813 A1
(43) Pub. Date: Mar. 15, 2018

(54) Electronic Devices With Protective Enzymes

(71) Applicant: Apple Inc., Cupertino, CA (US)

(72) Inventors: Simon R. Lancaster-Larocque, San Jose, CA (US); Paul Choiniere, Livermore, CA (US); Dinesh C. Mathew, San Francisco, CA (US); Patrick Magannig Boyle, Boston, MA (US)

(21) Appl. No.: 15/702,648

(22) Filed: Sep. 12, 2017

Related U.S. Application Data
(60) Provisional application No. 62/393,501, filed on Sep. 12, 2016.

(51) Int. Cl. H05K 5/06; C12N 9/02; C09K 3/10; C09J 7/02; C12N 11/08; H05K 5/00; H05K 5/04

(52) U.S. Cl. CPC H05K 5/06; C12N 9/0069; C09K 3/10; C09J 7/02; C12N 11/08; C09K 2200/0482; H05K 5/04; C09J 2201/128; C09J 2203/326; C09J 2489/00; C09J 2489/001; H05K 5/0017

(57) ABSTRACT

An electronic device may have polymer structures such as polymer electronic device structures, gaskets for forming seals between display cover layers and housing structures, polymer structures that fill gaps between housing walls, adhesive, adhesive tapes, and other structures with polymer. To prevent degradation of the polymer from exposure to fatty acids and other potentially harmful materials, a protective enzyme may be incorporated into one or more of the polymer structures. The protective enzyme may be a lipoxygenase enzyme or other enzyme that degrades harmful substances such as fatty acids and thereby neutralizes the harmful substances and prevents damage to the polymer structures.

This application claims the benefit of provisional patent application No. 62/393,501, filed on Sep. 12, 2016, which is hereby incorporated by reference herein in its entirety.

### FIELD

This relates generally to electronic devices and, more particularly, to preventing degradation of polymer structures in electronic devices.

### BACKGROUND

Electronic devices often include polymers. For example, electronic device housing structures, gaskets, adhesives, and other structures may be formed from polymers. If care is not taken, these structures may be vulnerable to degradation from environmental contaminants. For example, exposure to fatty acids from human sweat and skin oils can cause polymers to swell and degrade.

### SUMMARY

An electronic device may have polymer structures such as polymer housing structures, gaskets for forming seals between structures such as display cover layers and housing structures, polymer that fills gaps between housing walls, adhesive, tapes, and other structures with polymer. To prevent degradation of the polymer from exposure to fatty acids and other potentially harmful materials, a protective enzyme may be incorporated into one or more of the polymer structures.

The protective enzyme may be a lipoxygenase enzyme or other enzyme that degrades harmful substances such as fatty acids and thereby neutralizes the harmful substances and prevents damage to the polymer structures.

The present disclosure encompasses the recognition that certain enzymes and polypeptides may be useful to neutralize (e.g., prevent or mitigate) damage to polymeric compositions caused by lipids. Exposure to certain secreted biological fluids containing lipids (e.g., human sweat and skin oils) can cause some polymers to swell and degrade. In some embodiments, an enzyme of the present disclosure facilitates degradation of a lipid, and thereby prevents damage to a polymeric structure. In some embodiments, an enzyme useful in the context of the present disclosure facilitates degradation of harmful lipids such as fatty acids.

The present disclosure provides methods and compositions that protect components of a composition (e.g., polymers) from damage by lipids (e.g., fatty acids) and/or other compounds that may cause damage. In some embodiments, a lipid and/or other compound that may cause damage is present in a biological fluid. In some embodiments methods and compositions are provided that include protective enzymes useful for breakdown of components in biological fluids that may cause damage to a polymer. In some embodiments, methods and compositions are provided where one or more protective enzymes are embedded within a composition. In some embodiments, methods and compositions are provided where one or more protective enzyme are applied to the surface of a composition (e.g., as a coating).

In some embodiments, methods are provided for promoting breakdown of one or more lipids (e.g., fatty acids) in a biological fluid, the method comprising contacting the biological fluid with an enzyme. In some embodiments, a method for promoting breakdown of one or more lipids in a biological fluid comprises contacting the biological fluid with a plurality of enzymes.

In some embodiments, methods are provided for inhibiting swelling of a polymer caused by exposure to a fatty acid, the methods comprising contacting the polymer with an enzyme. In some embodiments, methods comprise contacting the polymer with a plurality of enzymes. In some embodiments, methods comprise embedding a plurality of enzymes within a polymer. In some embodiments, methods comprise coating a polymeric composition with a plurality of enzymes.

In some embodiments, methods are provided for inhibiting swelling of a polymer caused by exposure to a fatty acid, the method comprising contacting the polymer with a polypeptide that binds the fatty acid. In some embodiments, a polypeptide forms a complex with the fatty acid. In some embodiments, polypeptide binding to a fatty acid inhibits reactivity of the fatty acid. In some embodiments, a polypeptide binds to a fatty acid, and thereby prevents it from damaging a polymer. In some embodiments, a polypeptide inhibits diffusion of the fatty acid.

In some embodiments, compositions are provided comprising an enzyme embedded in a polymer. In some embodiments, a composition comprises two or more enzymes embedded in a polymer. In some embodiments, compositions comprise a polymer and two or more enzymes that promote breakdown of a fatty acid.

In some embodiments, compositions are provided comprising a polymer and one or more enzymes that promote breakdown of a fatty acid and one or more polypeptides that form a complex with a fatty acid. In some embodiments, a polypeptide binding to a fatty acid inhibits the reactivity of the fatty acid. In some embodiments, a polypeptide binds to a fatty acid, and thereby prevents it from damaging a polymer. In some embodiments, a polypeptide inhibits diffusion of the fatty acid.

In some embodiments, topical formulations are provided comprising one or more enzymes that promote breakdown of a fatty acid. In some embodiments, topical formulations are provided comprising one or more polypeptides that form a complex with a fatty acid.

In some embodiments, compositions are provided comprising one or more enzymes that promote breakdown of a fatty acid and one or more polypeptides that form a complex with a fatty acid. In some embodiments, polypeptide binding to a fatty acid inhibits the reactivity of the fatty acid. In some embodiments, a polypeptide binds to a fatty acid, thereby prevent it from damaging a polymer. In some embodiments, a polypeptide inhibits diffusion of the fatty acid.

In some embodiments, a biological fluid is secreted by a human. In some embodiments, a biological fluid is or comprises human sweat. In some embodiments, a biological fluid is or comprises sebum. In some embodiments, a lipid in a biologic fluid is an unsaturated fatty acid. Unsaturated fatty acids include, for example, palmitoleic acid, oleic acid, myristoleic acid, linoleic acid, and arachidonic acid. In some embodiments, a fatty acid is an oleic acid. In some embodiments, a fatty acid is a linoleic acid. In some embodiments, a biological fluid comprises a plurality of fatty acids to be neutralized.

In some embodiments, enzymes for use in a method or composition of the present disclosure include dioxygenases, monooxygenases, heme peroxidases, P450s, and combinations and/or variants thereof. In some embodiments, an enzyme is a dioxygenase. In some embodiments, an enzyme is a monooxygenase. In some embodiments, methods comprise contacting the bodily fluid with a plurality of enzymes.

In some embodiments, an enzyme is of animal origin. In some embodiments, an enzyme is of plant origin. In some embodiments, an enzyme is of fungal origin. In some embodiments, an enzyme is of bacterial origin. In some embodiments, an enzyme is a cyanobacterial enzyme. In some embodiments, an enzyme is from archaea.

In some embodiments, an enzyme catalyzes beta or omega oxidation. In some embodiments, an enzyme catalyzes hydroperoxidation at the 10S and/or 12S-carbon of a fatty acid (e.g., an oleic acid). In some embodiments, an enzyme for use in the context of the present disclosure does not require adenosine triphosphate (ATP) for catalytic activity.

In some embodiments, an enzyme is a lipoxygenase. In some embodiments, a lipoxygenase has 10S-LOX activity. In some embodiments, a 10S-LOX is or comprises a sequence of any one of SEQ ID NOs:1, 2, and 3. In some embodiments, a 10S-LOX enzyme has a sequence that is at least about 50% (e.g., at least about 55%, 60%, 65%, 70%, 75%, 80%, 85%, 90%, 95%, 96%, 97%, 98%, or 99%) identical to any one of SEQ ID NOs: 1, 2, and 3. In some embodiments, an enzyme is a lipoxygenase that catalyzes hydroperoxidation at positions 9 and/or 13 of a fatty acid. In some embodiments, an enzyme is a lipoxygenase that catalyzes hydroperoxidation at positions 9S and/or 13S of a fatty acid. In some embodiments, an enzyme is a lipoxygenase that catalyzes hydroperoxidation at positions 9R and/or 13R of a fatty acid.

In some embodiments, an enzyme is embedded in a polymer. In some embodiments, an enzyme is applied to the surface of a polymer. In some embodiments, a polymer is a component of a device. In some embodiments, the device is an electronic device.

### BRIEF DESCRIPTION OF THE DRAWINGS

FIG. 1 is a cross-sectional side view of an illustrative electronic device in accordance with an embodiment.

FIG. 2 is a diagram showing how structures in an electronic device may be attached to each other in accordance with an embodiment.

FIG. 3 is a cross-sectional side view of an illustrative joint between electronic device structures that has been formed using double-sided tape and a gasket in accordance with an embodiment.

FIG. 4 is a cross-sectional side view of an illustrative electronic device having structures such as display and housing structures sealed using a gasket in accordance with an embodiment.

FIG. 5 depicts swelling of a representative adhesive upon exposure to an exemplary fatty acid, oleic acid. Y-axis indicates the amount of swelling by area, X-axis indicates the number of hours post-exposure.

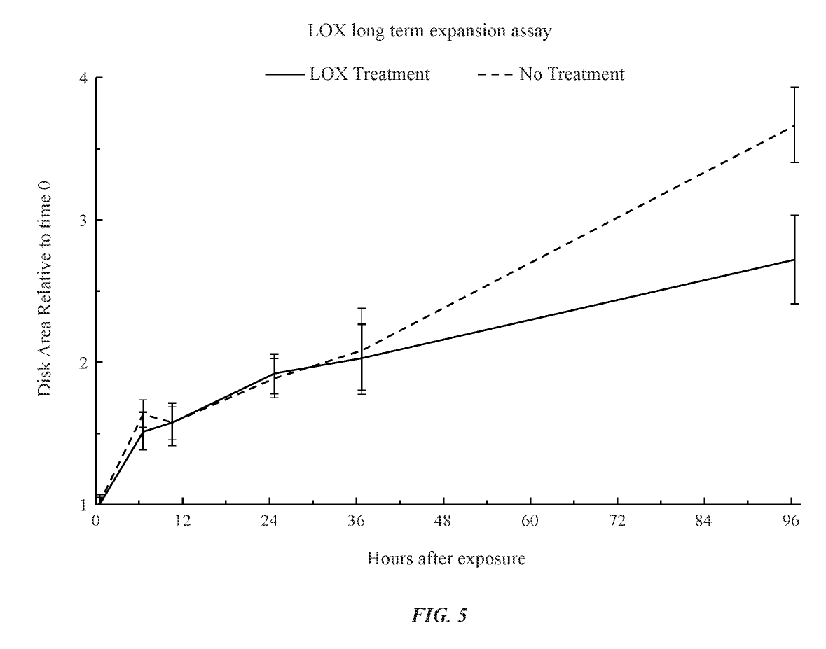

FIG. 6 is a schematic illustrating relatedness of certain enzymes for 10S-LOX activity.

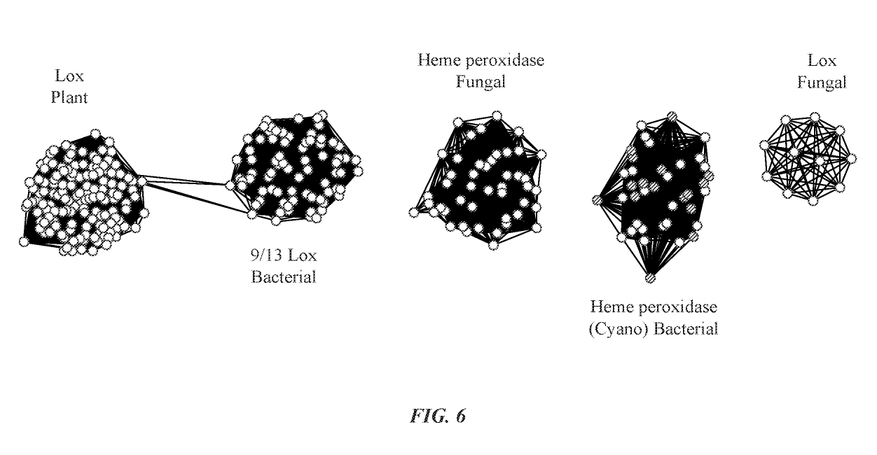

### DETAILED DESCRIPTION

Electronic devices may be exposed to the bodily fluids of users such as sweat and sebum. These bodily fluids may contain fatty acids that degrade polymers in the electronic devices. To prevent degradation, protective enzymes may be incorporated into the electronic devices. The protective enzymes may include, for example, one or more enzymes from the lipoxygenase (LOX) enzyme family, or other suitable enzymes that degrade harmful substances such as fatty acids.

A protective enzyme may be incorporated into a coating, an adhesive, a gasket, or other structures in an electronic device. When fatty acids come into contact with a protective enzyme in a coating, adhesive, gasket, or other structure, the fatty acids in the structures are neutralized.

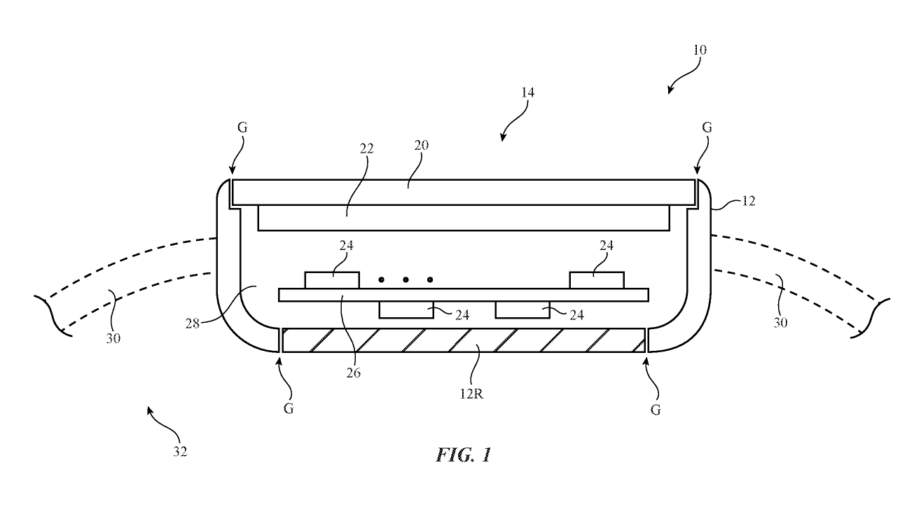

FIG. 1 is a cross-sectional view of an illustrative electronic device of the type that may include one or more structures with a protective enzyme. Electronic device 10 may be a computing device such as a laptop computer, a computer monitor containing an embedded computer, a tablet computer, a cellular telephone, a media player, or other handheld or portable electronic device, a smaller device such as a wrist-watch device, a pendant device, a headphone or earpiece device, a device embedded in eyeglasses or other equipment worn on a user's head, or other wearable or miniature device, a television, a computer display that does not contain an embedded computer, a gaming device, a navigation device, an embedded system such as a system in which electronic equipment with a display is mounted in a kiosk or automobile, equipment that implements the functionality of two or more of these devices, an accessory (e.g., earbuds, a remote control, a wireless trackpad, etc.), or other electronic equipment. In the illustrative configuration of FIG. 1, device 10 is a portable device such as a wristwatch or cellular telephone. Other configurations may be used for device 10 if desired. The example of FIG. 1 is merely illustrative.

As shown in FIG. 1, device 10 may include display 14. Display may be mounted in housing 12. Display 14 may have a rectangular outline (footprint when viewed from above) and housing 12 may as a correspondingly rectangular footprint. Other shapes may be used for display 14 and housing 12 if desired.

Housing 12, which may sometimes be referred to as an enclosure or case, may be formed from plastic, glass, ceramics, fiber composites, metal (e.g., stainless steel, aluminum, etc.), other suitable materials, or a combination of any two or more of these materials. Housing 12 may be formed using a unibody configuration in which some or all of housing 12 is machined or molded as a single structure or may be formed using multiple structures (e.g., an internal frame structure, one or more structures that form exterior housing surfaces, etc.). Openings may be formed in housing 12 to form communications ports, holes for buttons, and other structures.

Display 14 may be a touch screen display that incorporates a layer of conductive capacitive touch sensor electrodes or other touch sensor components (e.g., resistive touch sensor components, acoustic touch sensor components, force-based touch sensor components, light-based touch sensor components, etc.) or may be a display that is not touch-sensitive. Capacitive touch sensor electrodes may be formed from an array of indium tin oxide pads or other transparent conductive structures.

Display 14 may be protected using display cover layer 20. Display cover layer 20 may be formed from a clear layer such as such as a layer of transparent glass, clear plastic, transparent ceramic, sapphire or other transparent crystalline material, or other transparent layer(s). Display cover layer 20 may have a planar shape, a convex curved profile, a concave curved profile, a shape with planar and curved portions, a layout that includes a planar main area surrounded on one or more edges with a portion that is bent out of the plane of the planar main area, or other suitable shape. Openings may be formed in the display cover layer to accommodate buttons, speaker ports, and other components.

Display 14 may have a display module such as display module 22 (sometimes referred to as a display layer, display, display unit, display structures, pixel array, etc.). Display module 22 may be a liquid crystal display, an organic light-emitting diode display, a pixel array formed from an array of crystalline semiconductor light-emitting diode dies, a plasma display, an electrophoretic display, a microelectromechanical systems display, or other suitable display.

If desired, a strap such as wrist strap 30 may be attached to housing 12. Strap 30 may be used to attach device 10 to the wrist of a user. Strap 30 may be formed from metal, plastic, leather, or other materials. If desired, strap 30 may be omitted (e.g., in configurations in which device 10 is too large to comfortably wear on a user's wrist).

Device 10 may include control circuitry formed from components 24 (e.g., integrated circuits) mounted on one or more substrates such as substrate 26 (e.g., a printed circuit board). The control circuitry may include processing circuitry such as one or more microprocessors, microcontrollers, digital signal processors, baseband processor integrated circuits, application specific integrated circuits, etc. and may include non-volatile storage (e.g., flash memory or other electrically-programmable-read-only memory configured to form a solid state drive), volatile storage (e.g., static or dynamic random-access-memory), etc. Components 24 may also include input-output devices such as buttons, touch sensors, microphones, speakers, sensors, etc. During operation, the control circuitry and input-output circuitry formed from components in device 10 such as components 24 may be used to gather input from a user and/or the environment and may be used to provide a user with output (e.g., images on display 14, sound output, etc.).

Components 24 may be mounted in interior 28 of housing 12 and device 10. Housing 12 and display 14 (e.g., display cover layer 20) may help prevent moisture from intruding into interior 28 from the ambient environment surrounding device 10 (i.e., exterior region 32). Device 10 may include gaps such as gaps G at interfaces between housing 12 and display 14, between portions of housing 12, and between other structures in device 10.

Consider, as an example, a scenario in which housing 12 includes a rear wall such as rear wall 12R. As shown in FIG. 1, gaps G may be formed between rear wall 12R and adjacent portions of housing 12. Rear wall 12R may include transparent members (e.g., transparent windows that allow light from light-emitting diodes in interior 28 to pass from interior 28 to exterior 32 and that allow exterior light to be received by light sensors in interior 28). If desired, rear wall 12R may include other sensors and/or input-output components, may be configured to form an antenna window, may be configured to form a lid for a battery compartment, or may be used to form other structures that are mounted within adjacent portions of housing 12. These structures may be formed from metal, plastic, glass, ceramic, etc. Only one rear wall portion 12R is shown in FIG. 1, but multiple separate members such as illustrative wall portion 12R of FIG. 1 may be formed in device 10, if desired.

Adhesive, gaskets, tape, and other structures (e.g., structures formed from polymers) may be formed in gaps G. These polymer structures may be vulnerable to damage from fatty acids.

Sebum and sweat contain fatty acids such as oleic acid. Oleic acid and other fatty acids (e.g., unsaturated fatty acids or other fatty acids) may include reactive compounds such as peroxide that can damage polymers. Protective enzymes such as lipoxygenase enzymes can degrade these reactive compounds (e.g., peroxide) and thereby reduce the ability of the fatty acid (e.g., oleic acid, etc.) to harm electronic device polymers. The protective enzymes neutralize the destructive activity of the fatty acids and thereby help enhance robustness of polymer structures for electronic devices.

In configurations for device 10 such as the configuration of FIG. 1 in which one or more separate housing members such as housing wall portion 12R are mounted to remaining portions of housing 12, gaps G may be present between rear wall portion 12R and the remaining portions of housing 12. Gaps G may also be present between display 14 and housing 12 (e.g., between display cover layer 20 and housing 12). These areas may be sealed using polymer structures (adhesive, gaskets, etc.) and may represent vulnerable locations for moisture intrusion. It may therefore be helpful to incorporate a protective enzyme into such structures and/or the gaps G between structures. A protective enzyme may also be incorporated into other structures (e.g., a plastic rear wall structure such as wall 12R, a plastic button member, etc.).

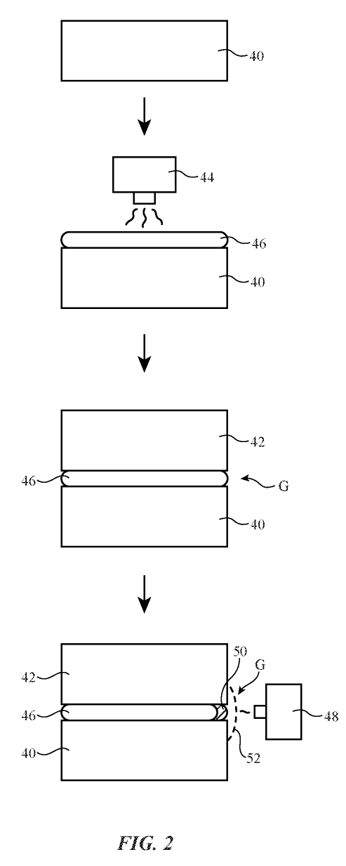

FIG. 2 is a diagram showing arrangements for incorporating protective enzyme into device. In the example of FIG. 2, device structures 40 and 42 are being joined. Structures 40 and/or 42 may be housing structures (e.g., housing 12, rear wall 12R, other portions of a device housing), may be portions of display 14 (e.g., display cover layer 20), may be button structures, may be portions of a microphone port, speaker port, or connector port, and/or may be other portions of device 10. As shown in FIG. 2, adhesive dispensing equipment 44 may dispense adhesive layer 46 (e.g., liquid adhesive, pressure sensitive adhesive, or other adhesive) onto structure 40. Dispensing equipment 44 may include spraying equipment, printing equipment (ink-jet printing equipment, pad printing equipment, screen printing equipment, etc.), equipment for applying adhesive by dipping or dripping, needle dispensing equipment, and/or other adhesive coating equipment. Adhesive 46 may be formed acrylic, silicone, polyurethane, epoxy, or other suitable polymers. Adhesive 46 may be heat-cured adhesive, adhesive that is cured by application of catalyst (e.g. liquid catalyst), ultraviolet-light-cured adhesive, room-temperature-cured adhesive, etc.

Structure 42 may be attached to structure 40 using adhesive 46 (e.g., by pressing structures 40 and 42 together using computer-controlled assembly equipment and/or manually). This forms a gap sealing joint of adhesive 46 between structures 40 and 42 and thereby helps to seal gap G between structures 40 and 42. Additional dispensing equipment (see, e.g., dispensing equipment 48) may, if desired, apply one or more additional materials at gap G. These materials may include, for example, material 50 and material 52. Material 50 may have a low viscosity and may wick into the gap G between structures 42 and 40 or may have a higher viscosity. Material 52 may cover gap G and material 50 and may therefore serve as a coating layer.

A protective enzyme may be incorporated into structures 40 and/or 42 (e.g., molded and/or machined plastic members formed from polycarbonate, acrylic, and/or other polymers), may be incorporated into adhesive 46, may be incorporated into material 50, and/or may be incorporated into material 52.

The protective enzyme may, for example, be incorporated into structures 40 and/or 42. In this type of arrangement, potentially harmful substances such as fatty acids that enter gap G will come into contact with the adjacent surfaces of structures 40 and/or 42 and will be degraded by exposure to these surfaces.

The protective enzyme may also be incorporated into adhesive 46. This may help directly protect adhesive 46 from materials such as fatty acids. By preventing degradation of adhesive 46 from exposure to fatty acids and other potentially harmful substances, adhesive 46 may form a robust connection and seal between structures 40 and 42.

If desired, protective enzyme may be incorporated into material 50. Material 50 may be, for example, a polymer carrier material or other material that helps dispense and adhere the protective enzyme to adhesive 46 and/or structures 40 and/or 42 (with or without adding structural strength to the joint formed by adhesive 46). In this type of configuration, material 50 may serve as a protective enzyme barrier (e.g., a protective gap filler). Fatty acids and other potentially harmful substances that enter gap G will be degraded by the protective enzyme in material 50 before reaching adhesive 46, so the presence of the protective enzyme in material 50 may help protect adhesive 46 from degradation. By incorporating the protective enzyme into material 50, concerns about compatibility between the protective enzyme and the materials and fabrication processes associated with forming structures 40, 42, and adhesive 46 may be avoided. If desired, a coating of material 50 may be formed on the surface of a plastic member such as a housing member, button, window structure, etc. The configuration of FIG. 2 in which material 50 is being used to fill gap G is merely illustrative.

Material 52 may form a protective coating layer that helps prevent potentially harmful materials from reaching material 50 and adhesive 46. If desired, material 52 may include a protective enzyme. For example, material 52 may be a polymer material that contains a protective enzyme. In arrangements in which material 52 includes protective enzyme, the protective enzyme may degrade fatty acids and other potentially harmful substances that come into contact with material 52 before these substances come into contact with material 50, thereby helping to prevent degradation of material 50 and adhesive 46. If desired, protective enzyme may be omitted from material 52 (e.g., in configurations in which material 50 contains protective enzyme). Material 52 may, for example, be a hydrophobic material such as a fluoropolymer that helps repel moisture. Material 52 may cover material 50 to help protect material 50 from moisture and, if desired, may cover additional portions of device 10 such as portion of the surfaces of structures 40 and 42.

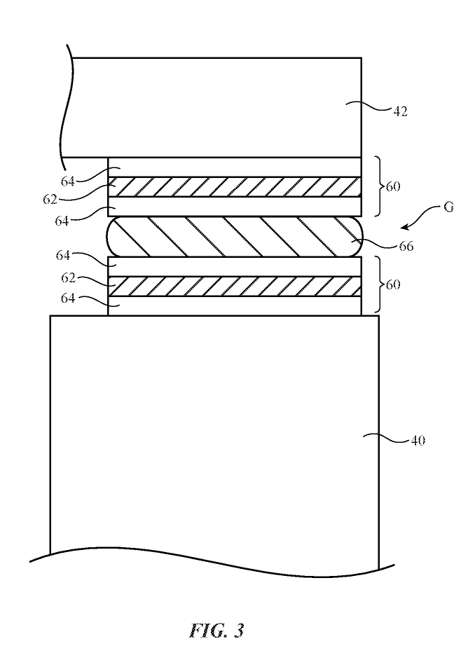

FIG. 3 is a cross-sectional side view of an illustrative arrangement for attaching structures 40 and 42 using layers of double-sided adhesive tape (tape 60). Each tape layer 60 may include a polymer carrier film (e.g., a flexible layer of polyethylene terephthalate or other polymer) such as polymer carrier film 62. Each polymer carrier film 62 may have a pair of opposing surfaces that are coated with respective layers of adhesive 64 (e.g., pressure sensitive adhesive). A tape layer 60 may be used to couple structures 40 and 42 together or two tape layers may be used to couple structures 40 and 42 to a structures that are interposed between structures 40 and 42 in gap G such as gasket 66. Gasket 66 may, for example, be formed from an elastomeric polymer such as silicone or other polymer.

To prevent degradation to the sealing provided by the structures in gap G of FIG. 3, a protective enzyme may be incorporated into tape 60 (e.g., into polymer carrier film 62 and/or adhesive layers 64). If desired, a protective enzyme may also be incorporated into structures 40 and/or 42, and/or may be incorporated into gasket 66. Gap filling and coating arrangements of the type described in connection with materials 50 and 52 of FIG. 2 may also be used.

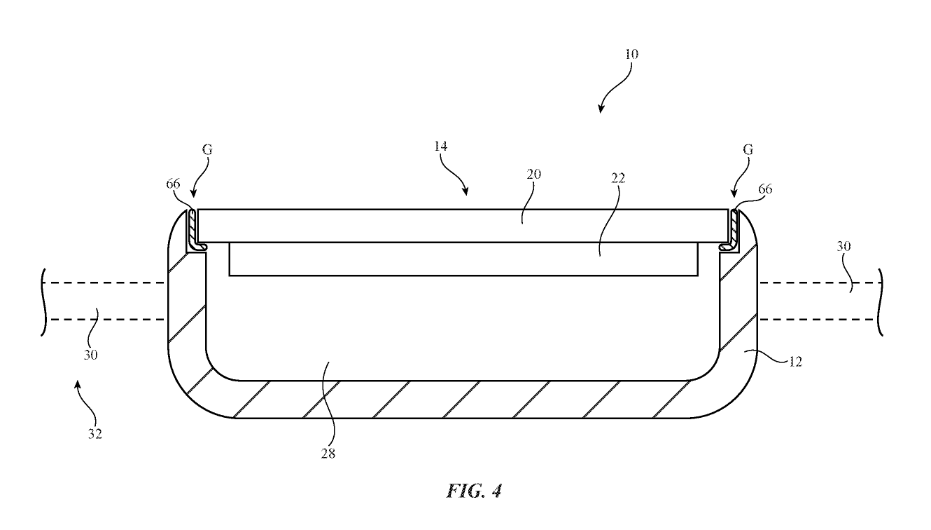

In the example of FIG. 4, gasket 66 is being used to form a seal in gap G between display cover layer 20 and housing 12. Gasket 66 of FIG. 4 may include a protective enzyme and/or may be covered with one or more additional layers to help prevent degradation to gasket 66 due to exposure to fatty acids and other potentially harmful compounds. For example, a protective enzyme may be dispensed into gap G as part of material 50 of FIG. 2 after mounting display cover layer 20 in housing 12 and, if desired, a hydrophobic coating or other coating layer (see, e.g., coating layer 52 of FIG. 2) may be dispensed onto material 50 and gasket 66. Adhesive 46 may also be incorporated into gaps G between display cover layer 20 and housing 12 of FIG. 4.

In general, any suitable components in device 10 may include a lipoxygenase enzyme or other protective enzyme(s). For example, a protective enzyme may be incorporated into plastic portions of housing 12, gaskets, adhesive layers, tape layers, coatings, gap filling sealant and other sealants, liquid polymers that are dispensed as coatings, room temperature adhesives, fluoropolymer coatings and/or other hydrophobic coatings, liquid polymer materials that serve as carrier fluids for enzyme dispensing without serving as structural adhesive, and/or other materials (e.g., polymers) in device 10. The foregoing embodiments are presented as examples.

In order that the present invention may be more readily understood, certain terms are defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Designed: As used herein, the term “designed” refers to an agent (i) whose structure is or was selected by the hand of man; (ii) that is produced by a process requiring the hand of man; and/or (iii) that is distinct from natural substances and other known agents.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Enzyme: As used herein, an “enzyme” is a molecule that catalyzes one or more biochemical reactions. In some embodiments, an enzyme is or comprises a polypeptide and/or RNA. In some embodiments, an enzyme is a polypeptide. In some embodiments, an enzyme is a polypeptide and that ranges from about 50 amino acid residues to 2,500 amino acid residues. Enzymes can be classified according to the reaction they catalyze. In some embodiments, enzymes include oxidoreductases (e.g., catalyze oxidation/reduction reactions), transferases (e.g., transfer a functional group, such as, for example, a methyl or phosphate group), hydrolases (e.g., catalyze hydrolysis of various bonds), lyases (e.g., cleave various bonds by means other than hydrolysis and oxidation), isomerases (e.g., catalyze isomerization changes within a single molecule) and ligases (e.g., join two molecules with covalent bonds). In some embodiments, a member of an enzyme class or family shows significant sequence homology or identity with, shares a common sequence motif (e.g., a characteristic sequence element) with, and/or shares a common activity (in some embodiments at a comparable level or within a designated range) with a reference enzyme of the class; in some embodiments with all enzymes within the class). For example, in some embodiments, a member enzyme shows an overall degree of sequence homology or identity with a reference enzyme that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, an enzyme suitable for use in the context of the present disclosure is of animal origin. In some embodiments, an enzyme suitable for use in the context of the present disclosure is of plant origin. In some embodiments, an enzyme suitable for use in the context of the present disclosure is of fungal origin. In some embodiments, an enzyme suitable for use in the context of the present disclosure is of bacterial origin.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions).

Human: In some embodiments, a human is an embryo, a fetus, an infant, a child, a teenager, an adult, or a senior citizen.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise D-amino acids, L-amino acids, or both. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a useful polypeptide may comprise or consist of a fragment of a parent polypeptide. The term “peptide” is generally used to refer to a polypeptide having a length of less than about 100 amino acids, less than about 50 amino acids, less than 20 amino acids, or less than 10 amino acids.

Recombinant: As used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source.

### Enzymes

In some embodiments, provided herein are enzymes useful for breakdown of compounds (e.g., fatty acids) that may cause damage to components of a composition (e.g., polymers). In some embodiments, enzymes for use in the context of the present disclosure include dioxygenases, monooxygenases, heme peroxidases, P450s, and combinations and/or variants thereof. In some embodiments, each of the enzymes can degrade an unsaturated fatty acid. In some embodiments, an enzyme specifically degrades one or more unsaturated fatty acids.

In some embodiments, compositions and methods of the present disclosure comprise two or more enzymes that promote breakdown of compounds that cause damage. In some embodiments, two or more enzymes each promote breakdown of unsaturated fatty acids.

In some embodiments, compositions and methods of the present disclosure comprise a plurality of enzymes. In some embodiments, each of the enzymes can degrade an unsaturated fatty acid. In some embodiments, each of the plurality of enzymes exhibit different substrate specificity for one or more unsaturated fatty acids.

In some embodiments, an enzyme is of animal origin. In some embodiments, an enzyme is of plant origin. In some embodiments, an enzyme is of fungal origin. In some embodiments, an enzyme is of bacterial origin. In some embodiments, an enzyme is a cyanobacterial enzyme. In some embodiments, an enzyme is from archaea.

In some embodiments, an enzyme catalyzes beta or omega oxidation. In some embodiments, an enzyme catalyzes beta or omega oxidation of fatty acids. In some embodiments, an enzyme has activity at the 5, 8, 9, 10, 11, 12, 13 or 15-carbon of a fatty acid. In some embodiments, an enzyme has activity at a 5R, 5S, 8R, 8S, 9R, 9S, 10S, 11R, 11S, 12R, 12S, 13R, 13S and/or 15S position of a fatty acid. In some embodiments, an enzyme has activity at the 10S and/or 12S-carbon of a fatty acid (e.g., an oleic acid). In some embodiments, an enzyme for use in the context of the present disclosure does not require adenosine triphosphate (ATP) for catalytic activity.

In some embodiments, a polymeric composition comprises an enzyme within a range from about 0.0001% to about 20% on w/w basis. In some embodiments, a polymeric composition comprises an enzyme within a range bounded by a lower limit and an upper limit, the upper limit being larger than the lower limit. In some embodiments, the lower limit may be about 0.0001%, 0.0002%, 0.0005%, 0.001%, 0.002%, 0.005%, 0.01%, 0.02%, 0.05%, 0.1%, 0.2%, 0.5%, 1%, 2%, 5%, or 10%. In some embodiments, the upper limit may be about 0.0005%, 0.001%, 0.002%, 0.005%, 0.01%, 0.02%, 0.05%, 0.1%, 0.2%, 0.5%, 1%, 2%, 5%, 10%, or 20%.

### Lipoxygenases

In some embodiments, an enzyme for use in the context of the present disclosure is a lipoxygenase. Lipoxygenase (LOX) enzymes catalyze oxygen-dependent oxidation of fatty acid substrates (for example, linoleic acid and arachidonic acid) to form hydroperoxy-fatty acid products. LOX enzymes are categorized as dioxygenases. Certain LOX enzymes have been purified from diverse organisms that display a broad range of substrate specificity and product specificity (e.g., site of oxidation within a fatty acid). LOX enzymes are widely expressed in animals, plants, and fungi, bacteria and cyanobacteria.

The present disclosure encompasses the recognition that lipoxygenases are a suitable class of enzymes for the degradation of unsaturated fatty acids (UFA) such as, for example, oleic acid. Without wishing to be bound by theory, LOX enzymes may convert unsaturated fatty acids to hydroperoxides, which can spontaneously degrade at the site of the double bond if not stabilized. In some embodiments, lipoxygenases may utilize either iron or manganese in their active sites.

In some embodiments, a LOX does not require ATP or other cofactors for catalytic activity. The present disclosure encompasses the recognition that activity independent of ATP or other cofactors is a desirable characteristic for certain applications.

In some embodiments, a lipoxygenase suitable for use in the context of the present disclosure is of animal origin. In some embodiments, a lipoxygenase suitable for use in the context of the present disclosure is of plant origin. In some embodiments, a lipoxygenase suitable for use in the context of the present disclosure is of fungal origin. In some embodiments, a lipoxygenase suitable for use in the context of the present disclosure is of bacterial origin. In some embodiments, a lipoxygenase suitable for use in the context of the present disclosure is of cyanobacterial origin. In some embodiments, a lipoxygenase suitable for use in the context of the present disclosure is from archaea.

In some embodiments, a LOX can catalyze hydroperoxidation of a fatty acid substrate. In some embodiments, a fatty acid substrate is an unsaturated fatty acid. In some embodiments, a LOX facilitates catalysis of palmitoleic acid, oleic acid, myristoleic acid, linoleic acid, and/or arachidonic acid.

In some embodiments, a LOX facilitates partial or complete degradation of one or more unsaturated fatty acids. In some embodiments, a LOX facilitates partial or complete degradation of palmitoleic acid, oleic acid, myristoleic acid, linoleic acid, and/or arachidonic acid.

In some embodiments, a LOX has activity (i.e., can catalyze hydroperoxidation) at the 5, 8, 9, 10, 11, 12, 13 or 15-carbon of a fatty acid. In some embodiments, a LOX has activity (i.e., can catalyze hydroperoxidation) at a 5R, 5S, 8R, 8S, 9R, 9S, 10S, 11R, 11S, 12R, 12S, 13R, 13S and/or 15S position of a fatty acid.

Bioprospecting identified LOX enzymes which act on oleic acid with a 10- or 12-carbon LOX preference. In some embodiments, a lipoxygenase catalyzes hydroperoxidation at the 10-carbon position of a fatty acid. In some embodiments, a lipoxygenase catalyzes hydroperoxidation at the 12-carbon position of a fatty acid. In some embodiments, a LOX catalyzes hydroperoxidation at the 10S and/or 12S-carbon of a fatty acid (i.e., has 10S-LOX or 12S-LOX activity).

In some embodiments, a lipoxygenase has 10S-LOX activity. In some embodiments, a lipoxygenase is a 10S-LOX from a plant, fungus, bacteria, or archaea. In some embodiments, a 10S-LOX facilitates catalysis of oleic acid. In some embodiments, a 10S-LOX is from cyanobacteria. In some embodiments, a 10S-LOX is from cyanobacteria and facilitates catalysis of oleic acid.

A common source of LOX is from plants (for example soybean), with specificity for an unsaturated fatty acid in which LOX activity occurs at the 9- or 13-carbon. In some embodiments, a lipoxygenase is a 9/13-LOX, for example a 9/13-LOX from plants, bacteria, archaea, or fungi.

The following sequences are representative of LOX enzymes that were characterized to have 10S-LOX activity.

In some embodiments, a 10S-LOX is or comprises a sequence any one of SEQ ID NOs: 1, 2, and 3. In some embodiments, a 10S-LOX enzyme has a sequence that is at least about 50% (e.g., at least about 55%, 60%, 65%, 70%, 75%, 80%, 85%, 90%, 95%, 96%, 97%, 98%, 99%, or 100%) identical to any one of SEQ ID NOs: 1, 2, and 3.

(SEQ ID NO: 1)
TRDTSRDGFSNKALAYTLTHFKPIWNLVQSYEPLKRKLNKFFLNSIIYKL
PTRPLPYSLMGLDPKIPGTDIPKKTDTYISWDSLTDKTYTGRHLPPDPEF
NKEGNLPPLDKVKTLFQKRDGKTIYSEKSTLLFPYWVQWFTDSFLRIDQE
NRFKNTSNHQIDMCNVYGLTRKQTNMLRAFKDGKFKTQKLKRKDGVEEEY
PLFYYADPEQGIIDPQFEGLHAPLNDEKRQPPEKKSKLFAMGVERANVQI
GYVMLNTLCIREHNRICDVLSKSYPEWDDERLFQTARNILMVIVLNIIME
EYIFHITPYNFRFFADPEAFTKESWYRENWMAIEFSFVYRWHSAIPETFI
YDGKEQSMYDSLWNNQMLIDKGLGALMEETCSQPGTRIGLFNTPDFKIAG
TPYTFIDATELASVKLGRQAQLASYNDYREMCGYPRVTDFNQITGDEYAQ
QKLKELYGHVDKIELFVGLYAEDVRKNSAIPPLVARIIGIDAFSQALTNP
LLSPKVFNKETFSEVGWEIIQNTKTVSDLVNRNVPPSDPKYKVSFEL
(SEQ ID NO: 2)
RDTSKDGFRNKLETYALTHFKPIWNLIQSNDTLKKKVNKFLVNNAIYKVP
TRPYPFSTMSPYTSWDSLSDRTYSGLHLPPLDWQPLTNENHLKLKLADTK
DFEKKLPAIEDLRGLYRKSGETKYSPKSTLIFPYFVQWFTDSFLRTDRHN
HRKNTSNHQIDLCTVYGLNAKITHLLRSYQGGKLKSQIINGEEYPPFYYD
EKGEAKKEFIGLPHQLDNDGNPKADTFPLDKKQKLFAMGVEVERSNVQIG
YVMLNVLALREHNRLCELLAKTYPSWDDERLFQTARNILIVEVLRIVVED
YVNHITPYHFQFITDPLTFSNEKWYRQNWMTVEFTLVYRWHSMLPDTLIY
NGQKIPTYETQWNNEMIIKQGLGALFEESCSQPCAQLSLFNTPEFLIPVE
LASVRFGREVKLRSYNDYRQLCKYPRVTDFDQISSDKNIQKELQRLYGHV
DNIELYVGIYAEDLRENSALPSLVGRLIGIDAFSQVLTNPLLAESVFHPE
TFSPVGWEEIQNTKTLSQLLHRNLPPSDKKYRVSFDRAST
(SEQ ID NO: 3)
AGKRDTSKDGFDNKVQTFLLTNFKGIWEIVQSNEFLKRKVNKTLINSLIY
KIPTRPNPYSMMTLDEYIPDTKIPKKTDTYTSWELLNDRTYIGRHLPPDP
KFNSEGNLPKVEDLAVLFRKRDGKTIYSPKSTMLFPYWVQWFTDSFLRID
HTKEKKLKNTSNHEIDLCNVYGLNRKRTHLLRTFKGGKFKTQKLKRQDGI
EEEYPLFYYADPAQGIVDPQFDGLYEPINDEKRLPADKKQYLFAMGVERA
NVQIGYVMLNTLCIREHNRLCDELASNYPDWDDERLFQTSRNILMAIILN
IIMEEYINHITPYHFKLFADPAAFVKESWYRPNWMTIEFDFVYRWHSAIP
ETFIYDGQPTDIAASLWNNKMFIDKGLGALMEETCSQPGTRIGLFNTPDI
LVELTELPSIRLGRQLQLASYNDYREMCGFPRVTKFEQITGDEFAQEKLK
ELYGHVDNIEFYVGLYAEEVRKNSTIPPLVARLIGIDAFSEALNNPLLSP
TIFNKDTFSPVGWEIIQNTKTVSDLINRNVPPSDKKYKVTFDL

A sequence similarity network of LOX enzymes is tested for activity on oleic acid. Green nodes showed 10S-LOX activity (FIG. 2). These sequences are distinct from 9/13-LOX from plants, bacteria, and fungi, and are sometimes annotated as heme peroxidases. In particular, the cluster contains primarily cyanobacterial enzymes contains the 10S-LOXs that are active on oleic acid.

In some embodiments, a fatty acid to be degraded is oleic acid, and a LOX enzyme specific for action on the 10S- or 12S-carbon of oleic acid is employed in a composition or method of the present disclosure.

### Dioxygenases and Monooxygenases

In some embodiments, dioxygenases other than LOX, as well as monooxygenases, are suitable for use in the context of the present disclosure. In some embodiments, an enzyme is a dioxygenase. In some embodiments, an enzyme is a monooxygenase.

In some embodiments, a dioxygenase catalyzes oxidation of a fatty acid substrate. In some embodiments, a fatty acid substrate is an unsaturated fatty acid. In some embodiments, a dioxygenase catalyzes oxidation of palmitoleic acid, oleic acid, myristoleic acid, linoleic acid, and/or arachidonic acid.

In some embodiments, a dioxygenase catalyzes the partial or complete degradation of one or more unsaturated fatty acids. In some embodiments, a dioxygenase facilitates partial or complete degradation of palmitoleic acid, oleic acid, myristoleic acid, linoleic acid, and/or arachidonic acid.

In some embodiments, a monooxygenase catalyzes oxidation of a fatty acid substrate. In some embodiments, a fatty acid substrate is an unsaturated fatty acid. In some embodiments, a monooxygenase catalyzes oxidation of palmitoleic acid, oleic acid, myristoleic acid, linoleic acid, and/or arachidonic acid.

In some embodiments, a monooxygenase catalyzes the partial or complete degradation of one or more unsaturated fatty acids. In some embodiments, a monooxygenase facilitates partial or complete degradation palmitoleic acid, oleic acid, myristoleic acid, linoleic acid, and/or arachidonic acid.

In some embodiments, a dioxygenase and/or a monooxygenase is from a plant, bacteria, archaea, or fungus. In some embodiments, a dioxygenase and/or a monooxygenase facilitates catalysis of oleic acid. In some embodiments, a dioxygenase and/or a monooxygenase is from cyanobacteria. In some embodiments, a dioxygenase and/or a monooxygenase is from cyanobacteria and facilitates catalysis of oleic acid.

In some embodiments, an enzyme catalyzes beta or omega oxidation. In some embodiments, an enzyme catalyzes oxidation or hydroperoxidation at the 5, 8, 9, 10, 11, 12, 13 or 15-carbon of a fatty acid. In some embodiments, an enzyme catalyzes oxidation or hydroperoxidation at a 5R, 5S, 8R, 8S, 9R, 9S, 10S, 11R, 11S, 12R, 12S, 13R, 13S and/or 15S position of a fatty acid. In some embodiments, an enzyme catalyzes oxidation or hydroperoxidation of the 10S and/or 12S-carbon of a fatty acid (e.g., an oleic acid). In some embodiments, an enzyme for use in the context of the present disclosure does not require adenosine triphosphate (ATP) for catalytic activity.

In some embodiments, a monooxygenase and/or dioxygenase in the context of the present disclosure can catalyze partial or complete degradation of a fatty acid (e.g., an unsaturated fatty acid). In some embodiments, activity of a monooxygenase and/or dioxygenase may be independent of cofactors (e.g., ATP). In some embodiments, activity of a monooxygenase and/or dioxygenase may require a cofactor (e.g., ATP).

In some embodiments, compositions and methods of the present disclosure comprise a LOX and one or more other dioxygenases that promote breakdown of compounds that cause damage to polymers. In some embodiments, compositions and methods of the present disclosure comprise a LOX and one or more monooxygenases that promote breakdown of compounds that cause damage to polymers. In some embodiments, compositions and methods of the present disclosure comprise a plurality of enzymes. In some embodiments, each of the enzymes can degrade an unsaturated fatty acid. In some embodiments, each of the plurality of enzymes exhibit different substrate specificity for one or more unsaturated fatty acids.

### P450s

In some embodiments, an enzyme suitable for use in the context of the present disclosure is a P450. Cytochrome P450 enzymes form a superfamily of hemoproteins found in bacteria, archaea and eukaryotes. In one of the most common activities, cytochrome P450 acts as a monooxygenase, by inserting one oxygen atom of molecular oxygen into a substrate molecule, while the other oxygen atom is reduced to water. A P450 catalytic reaction may require two electrons for the activation of molecular oxygen. P450s from eukaryotes use NADPH as the external reductant and source of electrons. Each electron may be transferred one at a time to a cytochrome P450 active site. In some embodiments, an electron transfer may be donated by an electron donor protein, e.g., a cytochrome P450 reductase (CPR). A CPR may be an electron donor protein for several different P450s from the same or from different organisms. In some cases P450s can also be coupled to a cytochrome b5 protein that can act as the electron donor protein or can improve the efficiency of the electron transfer from the CPR to the P450. In eukaryotic cells and particularly in plants, P450s and CPRs are generally membrane-bound proteins and are associated with the endoplasmic reticulum. These proteins may be anchored to the membrane by a N-terminal trans-membrane helix.

Many P450s have low substrate specificity and are therefore able to catalyze the oxidation of many diverse structures. Many P450s have a particular region and stereo-selectivity with a given substrate, however they produce a mixture of several products from a particular substrate. In some embodiments, a P450 is involved in breakdown and detoxification of molecules (e.g., xenobiotics). In some embodiments, a P450 is involved in biosynthetic pathways. P450s involved in biosynthetic pathways may exhibit specificity for certain types of substrates and region and stereo-selectivity. In some embodiments, a P450 is from a plant, bacteria, or fungus.

A large number of P450s can be found in nature and particularly in plants. One plant genome can contain several hundreds of genes encoding for P450s.

In some embodiments, a P450 is active on one or more unsaturated fatty acids. In some embodiments, a P450 facilitates catalysis of palmitoleic acid, oleic acid, myristoleic acid, linoleic acid, and/or arachidonic acid. In some embodiments, a P450 facilitates catalysis of oleic acid.

In some embodiments, compositions and methods of the present disclosure comprise a P450 and another enzyme (e.g., a dioxygenase, monooxygenase, heme peroxidase) that promote breakdown of compounds that cause damage. In some embodiments, compositions and methods of the present disclosure comprise a plurality of enzymes. In some embodiments, each of the enzymes can act on an unsaturated fatty acid. In some embodiments, each of the enzymes can degrade an unsaturated fatty acid. In some embodiments, each of the plurality of enzymes exhibit different substrate specificity for one or more unsaturated fatty acids.

### Heme Peroxidases

In some embodiments, an enzyme for use in the context of the present disclosure is a heme peroxidase. In some embodiments, a heme peroxidase has a ferriprotoporphyrin IX prosthetic group located at the active site. The plant enzymes horseradish peroxidase (HRP) and plant soyabean peroxidase (SBP) are examples of plant heme peroxidases.

In some embodiments, a heme peroxidase facilitates catalysis of one or more unsaturated fatty acids. In some embodiments, a heme peroxidase facilitates catalysis of palmitoleic acid, oleic acid, myristoleic acid, linoleic acid, and/or arachidonic acid. In some embodiments, a heme peroxidase facilitates catalysis of oleic acid.

In some embodiments, compositions and methods of the present disclosure comprise a heme peroxidase and another enzyme (e.g., a dioxygenase, monooxygenase, P450) that promotes breakdown of compounds that cause damage to polymers. In some embodiments, compositions and methods of the present disclosure comprise a plurality of enzymes. In some embodiments, each of the enzymes facilitates catalysis of an unsaturated fatty acid. In some embodiments, each of the enzymes can degrade an unsaturated fatty acid. In some embodiments, each of the plurality of enzymes exhibit different substrate specificity for one or more unsaturated fatty acids.

### Polypeptides

In some embodiments, protection of materials from damaging lipids (e.g., fatty acids, such as oleic acid and other unsaturated fatty acids), could additionally or alternatively be performed via non-catalytic binding of a protein to a lipid. In some embodiments, binding to a lipid (e.g., an unsaturated fatty acid) results in formation of a protein-lipid complex that would be less able to diffuse into the material being protected. Binding of a lipid (e.g., an unsaturated fatty acid) could also limit or prevent it from reacting with other materials, such as a polymeric material or other material to be protected.

In some embodiments, compositions are provided comprising one or more enzymes that promote breakdown of a fatty acid and one or more polypeptides that form a complex with a fatty acid.

### Nucleic Acid Construction and Expression

Enzymes and polypeptides as described herein may be produced from nucleic acid molecules using molecular biological methods known to the art. Nucleic acid molecules are inserted into a vector that is able to express the polypeptide when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the enzymes of the present disclosure under control of transcriptional/translational control signals. These methods may include in vitro recombinant DNA and synthetic techniques and in vivo recombination (See Sambrook et al., Molecular Cloning, A Laboratory Manual, Cold Spring Harbor Laboratory; Current Protocols in Molecular Biology, Eds. Ausubel, et al, Greene Publ. Assoc., Wiley-Interscience, NY).

Expression of nucleic acid molecules in accordance with the present disclosure may be regulated by a second nucleic acid sequence so that the molecule is expressed in a host transformed with the recombinant DNA molecule. For example, expression of the nucleic acid molecules of the present disclosure may be controlled by promoter and/or enhancer elements, which are known in the art.

Nucleic acid constructs of the present disclosure may be inserted into an expression vector or viral vector by methods known to the art, and nucleic acid molecules operatively linked to an expression control sequence.

Where appropriate, nucleic acid sequences that encode enzyme as described herein may be modified to include codons that are optimized for expression in a particular cell type or organism. Codon optimized sequences are synthetic sequences, and preferably encode the identical polypeptide (or a biologically active fragment of a full length polypeptide which has substantially the same activity as the full length polypeptide) encoded by the non-codon optimized parent polynucleotide. In some embodiments, the coding region of the genetic material encoding antibody components, in whole or in part, may include an altered sequence to optimize codon usage for a particular cell type (e.g., a eukaryotic or prokaryotic cell). For example, the coding sequence for an enzyme as described herein may be optimized for expression in bacterial cells, fungal cells, plant cells, mammalian cells, etc. Such a sequence may be described as a codon-optimized sequence.

An expression vector containing a nucleic acid molecule is transformed into a suitable host cell to allow for production of the protein encoded by the nucleic acid constructs. Exemplary host cells include prokaryotes (e.g., E. coli) and eukaryotes (e.g., yeast cells or mammalian cells). Host cells transformed with an expression vector are grown under conditions permitting production of an enzyme of the present disclosure followed by recovery of the enzyme or polypeptide.

Enzyme and polypeptides of the present disclosure may be purified by any technique, which allows for the subsequent formation of a stable enzyme. For example, not wishing to be bound by theory, enzymes may be recovered from cells either as soluble polypeptides or as inclusion bodies, from which they may be extracted quantitatively by 8M™ guanidinium hydrochloride and dialysis. In order to further purify enzymes of the present disclosure, conventional ion exchange chromatography, hydrophobic interaction chromatography, reverse phase chromatography or gel filtration may be used. Enzymes of the present invention may also be recovered from conditioned media following secretion from eukaryotic or prokaryotic cells.

### Polymers

Biological fluids may include components that can damage polymers. For example, fatty acids (e.g., unsaturated fatty acids or other fatty acids) may include reactive compounds such as peroxide that can damage polymers. The present invention encompasses the recognition that protective enzymes may be incorporated into a polymer to prevent, mitigate or lessen damage to a polymer and/or a device containing the polymer. The protective enzymes may include any enzymes described herein suitable to protect against biological substances (e.g., fatty acids) that may degrade or otherwise damage polymeric materials.

In some embodiments, polymers suitable for use in the context of the present disclosure may include polyamides, polyesters, polyaryletherketones, polyimides, polyetherimides, polyamideimide, liquid crystalline polymers, polycarbonates, polyolefins, polyphenylene oxide, polysulfones, polyacrylates, acrylonitrile butadiene styrene polymer, polyoxymethylene, polystyrene, polyarylene sulfide, polyvinylidene fluoride, polytetrafluoroethylene, polyvinylidene chloride, polyvinyl chloride, and any other suitable polymer. In some embodiments, a polymer is or comprises a silicone resin, epoxy resin, polyvinyl butyral resin, cellulose acetate, ethylene-vinyl acetate copolymer (EVA) or an ionomer. In some embodiments, a polymer is or comprises an acrylic-based polymer. In some embodiments, a polymer is or comprises a silicon-based polymer.

In some embodiments, a protective enzyme or polypeptide is embedded within a polymeric composition. In some embodiments, a polymeric composition comprises a protective enzyme or polypeptide within a range from about 0.0001% to about 20% on w/w basis. In some embodiments, a polymeric composition comprises two or more protective enzymes, each present within a range from about 0.0001% to about 20% on w/w basis. In some embodiments, a polymeric composition comprises a protective enzyme or polypeptide within a range bounded by a lower limit and an upper limit, the upper limit being larger than the lower limit. In some embodiments, the lower limit may be about 0.0001%, 0.0002%, 0.0005%, 0.001%, 0.002%, 0.005%, 0.01%, 0.02%, 0.05%, 0.1%, 0.2%, 0.5%, 1%, 2%, 5%, or 10%. In some embodiments, the upper limit may be about 0.0005%, 0.001%, 0.002%, 0.005%, 0.01%, 0.02%, 0.05%, 0.1%, 0.2%, 0.5%, 1%, 2%, 5%, 10%, or 20%.

In some embodiments, a protective enzyme is applied to the surface of a polymeric composition. In some embodiments, a polymeric composition is coated with a protective enzyme.

### Components of Electronic Devices

As described in connection with FIGS. 1, 2, 3, and 4, electronic devices may be exposed to the bodily fluids of users such as sweat and sebum. These bodily fluids may contain fatty acids that degrade polymers in electronic devices. To prevent degradation, protective enzymes may be incorporated into one or more components of electronic device. Protective enzymes may include, for example, one or more enzymes from the lipoxygenase (LOX) enzyme family, or other suitable enzymes that degrade harmful substances such as fatty acids. Protective enzymes such as lipoxygenase enzymes can degrade these reactive compounds (e.g., peroxide) and thereby reduce the ability of a fatty acid (e.g., oleic acid, etc.) to degrade polymers and damage electronic devices. Protective enzymes may neutralize the destructive activity of the fatty acids and thereby help enhance robustness of polymer structures for electronic devices.

In some embodiments, a polymer with a protective enzyme may be part of an adhesive, gasket, tape, button, or other structure that may be vulnerable to damage by exposure to biological lipids (e.g., fatty acids). A protective enzyme may be incorporated into a coating, an adhesive, a gasket, or other structures in an electronic device. When fatty acids come into contact with a protective enzyme in a coating, adhesive, gasket, or other structure, the fatty acids in the structures are neutralized.

In general, any suitable components in an electronic device may include one or more protective enzymes (e.g., dioxygenases, monooxygenases, heme peroxidases, P450s, and/or lipoxygenases). For example, a protective enzyme may be incorporated into plastic portions of housings, gaskets, adhesive layers, tape layers, coatings, gap filling sealant and other sealants, liquid polymers that are dispensed as coatings, room temperature adhesives, fluoropolymer coatings and/or other hydrophobic coatings, liquid polymer materials that serve as carrier fluids for enzyme dispensing without serving as structural adhesive, and/or other materials (e.g., polymers) in an electronic device.

### Topical Formulations

In some embodiments, a protective enzyme and/or polypeptide may be used as part of a topical formulation for application to the skin of a mammal (e.g., a human). In some embodiments, a protective enzyme and/or polypeptide may be used as part of a topical formulation for application to a polymer. In some embodiments, the polymer is a component of a device. In some embodiments, a protective enzyme and/or polypeptide may be used as part of a topical formulation for application to a device that comprises a polymer. Without wishing to be bound by theory, it is envisioned that inclusion of a protective enzyme and/or polypeptide may prevent degradation of other components of a topical formulation. In some embodiments, two or more protective enzymes may be used a part of a topical formulation for application to the skin of a mammal (e.g., a human). In some embodiments a mixture of enzymes may be used as part of a topical formulation. For example, it may be desirable to combine enzymes with specificities to multiple unsaturated fatty acids, for example to both oleic and linoleic acid present in sebum and sweat. Similarly, single enzymes or mixtures could be employed to target oleic acid or multiple unsaturated fatty acids in products applied to the skin.

In some embodiments, a topical formulation comprising a protective enzyme and/or polypeptide is an emulsion, gel, ointment, or lotion. Topical formulations may be prepared using methods known in the art, for example, as provided by reference texts such as, REMINGTON: THE SCIENCE AND PRACTICE OF PHARMACY 1577-1591, 1672-1673, 866-885; (Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference.

In some embodiments, a protective enzyme and/or polypeptide may be useful for compositions comprising a medicament for topical formulation. In some embodiments, a protective enzyme and/or polypeptide is a component of a topical sunscreen formulation.

In some embodiments, a topical formulation comprises a protective enzyme or polypeptide within a range from about 0.0001% to about 20% on w/w basis. In some embodiments, a polymeric composition comprises two or more protective enzymes, each present within a range from about 0.0001% to about 20% on w/w basis. In some embodiments, a topical formulation comprises a protective enzyme or polypeptide within a range bounded by a lower limit and an upper limit, the upper limit being larger than the lower limit. In some embodiments, the lower limit may be about 0.0001%, 0.0002%, 0.0005%, 0.001%, 0.002%, 0.005%, 0.01%, 0.02%, 0.05%, 0.1%, 0.2%, 0.5%, 1%, 2%, 5%, or 10%. In some embodiments, the upper limit may be about 0.0005%, 0.001%, 0.002%, 0.005%, 0.01%, 0.02%, 0.05%, 0.1%, 0.2%, 0.5%, 1%, 2%, 5%, 10%, or 20%.

The foregoing is merely illustrative and various modifications can be made to the described embodiments. The foregoing embodiments may be implemented individually or in any combination.

### EXAMPLES

### Example 1—an Exemplary Protective Enzyme Prevents Fatty Acid Induced Polymer Swelling

This example demonstrates the efficacy of an exemplary protective enzyme (e.g., a lipoxygenase). Specifically, this Example demonstrates that a polymer composition comprising an exemplary protective enzyme (e.g., a lipoxygenase) is able to prevent polymer swelling induced by treatment with a fatty acid. An acrylic foam pressure sensitive adhesive tape (3M™ VHB™ Tape 4914-015) was used as a representative composition to assess the efficacy protective enzymes to prevent polymeric swelling. This representative adhesive was exposed to an exemplary unsaturated fatty acid (oleic acid), and polymer swelling was assessed over time for both lipoxygenase treated (treated) and untreated samples, FIG. 3. Y-axis indicates the amount of swelling by area, X-axis indicates the number of hours post-exposure. As can be seen in FIG. 3, beginning at approximately 40 hours, the untreated samples show an increase in swelling greater than that of the treated samples. Thus, treatment with a lipoxygenase protected the representative adhesive from fatty acid induced damage over time.

### Example 2—Analysis of LOX Family Enzymes for Activity

This example describes assessment of a network of LOX enzymes for catalytic activity on an exemplary fatty acid substrate (e.g., oleic acid). Homologs of extant lipoxygenases were identified via bioinformatics methods, based on sequence and structural similarity. Each node represents a candidate LOX enzyme, with each enzyme clustered by pairwise similarity to the other enzymes in the library. Green nodes were shown to have 10S-LOX activity on oleic acid. These 10S-LOX enzymes distantly related to the more well-described 9S/13S-LOX enzymes described for plants and bacteria. To our knowledge, no 10S-LOX sequences have been described elsewhere. The above example demonstrates that lipoxygenase are a specific class of enzymes that can degrade an exemplary fatty acid, oleic acid. Moreover, we have determined that other enzymes, such as dioxygenases, monooxygenases, heme peroxidases, P450s, and others could also catalyze the degradation of lipids such as fatty acids.

Enzyme family networks are depicted in FIG. 4, with green nodes indicating possession 10S-LOX activity. Thus, several different heme peroxidase enzymes possess 10S-LOX activity. These sequences are distinct from 9/13-LOX from plants, bacteria, and fungi. Of note, the cluster contains primarily cyanobacterial enzymes was found to contain 10S-LOXs that are active on the specific exemplary fatty acid (oleic acid). The assays described herein can be used to characterize the ability of enzymes to protect from damage induced by lipids (e.g., specific fatty acids and/or combinations of fatty acids).

Having thus described at least several aspects and embodiments of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawing are by way of example only and the invention is described in further detail by the claims that follow.

The foregoing is merely illustrative and various modifications can be made to the described embodiments. The foregoing embodiments may be implemented individually or in any combination.

What is claimed is:

1. An electronic device, comprising:
a housing;
a display mounted in the housing; and
a polymer gasket in a gap between the housing and the display, wherein the polymer gasket includes a protective enzyme.

2. The electronic device defined in claim 1 wherein the protective enzyme comprises a lipoxygenase enzyme.

3. The electronic device defined in claim 2 wherein the display comprises a pixel array and a display cover layer that covers the pixel array and wherein the polymer gasket is interposed between the housing and the display cover layer.

4. An electronic device, comprising:
a first structure;
a second structure;
a gasket;
a first double-sided adhesive tape layer between the first structure and the gasket; and
a second double-sided adhesive tape layer between the second structure and the gasket, wherein the first and second double-sized adhesive tape layers include an enzyme.

5. The electronic device defined in claim 4 wherein the enzyme comprises a lipoxygenase enzyme.

6. The electronic device defined in claim 5 wherein the first structure comprises a housing structure.

7. The electronic device defined in claim 6 wherein the second structure comprises a display cover layer.

8. The electronic device defined in claim 7 further comprising a pixel array that is covered by the display cover layer, wherein the housing structure comprises a metal housing wall.

9. An electronic device, comprising:
a first structure;
a second structure;
a layer of adhesive that couples the first structure to the second structure; and
an enzyme that prevents degradation of the layer of adhesive.

10. The electronic device defined in claim 9 wherein the enzyme comprises a lipoxygenase enzyme.

11. The electronic device defined in claim 10 wherein the enzyme is in the adhesive.

12. The electronic device defined in claim 10 wherein the first and second structures are separated by a gap and wherein the layer of adhesive is in the gap, the electronic device further comprising a material in the gap adjacent to the layer of adhesive, wherein the enzyme is in the material that is in the gap adjacent to the layer of adhesive.

13. The electronic device defined in claim 12 further comprising a coating that covers the material that is in the gap adjacent to the layer of adhesive.

14. The electronic device defined in claim 13 wherein the coating comprises a fluoropolymer coating.

15. The electronic device defined in claim 10 wherein the first structure comprises an electronic device housing structure.

16. The electronic device defined in claim 15 wherein the second structure comprises an electronic device housing structure.

17. The electronic device defined in claim 10 further comprising a polymer material that is adjacent to the layer of adhesive, wherein the enzyme is in the polymer material and is not in the layer of adhesive.

18. The electronic device defined in claim 17 further comprising a hydrophobic coating that covers the polymer material.

19. The electronic device defined in claim 10 wherein the first structure comprises a polymer structure and wherein the enzyme is in the polymer structure.

20. The electronic device defined in claim 10 wherein the first and second structures comprise electronic device housing structures and wherein the enzyme is in the adhesive.
